(19) United States
(12) Patent Application Publication
(10) Pub. No.: US 2013/0228173 A1
(43) Pub. Date: Sep. 5, 2013

(54) PATIENT INTERFACE DEVICE WITH QUICK RELEASE MECHANISM

(71) Applicant: Lance Ranard Busch, Eindhoven (NL)

(72) Inventor: Lance Ranard Busch, Eindhoven (NL)

(73) Assignee: KONINKLIJKE PHILIPS ELECTRONICS N.V., EINDHOVEN (NL)

(21) Appl. No.: 13/988,105

(22) PCT Filed: Nov. 11, 2011
(86) PCT No.: PCT/IB2011/055041

§ 371 Date: May 17, 2013

Related U.S. Application Data
(60) Provisional application No. 61/416,317, filed on Nov. 23, 2010.

(51) Int. Cl. A61M 16/06

(52) U.S. Cl. CPC A61M 16/0683
USPC 128/202.27

(57) ABSTRACT

A patient interface device (8, 8′, 8″) that includes a cushion (16), a shell (18) coupled to the cushion, a headgear (22, 22′, 22″) having a first strap (28A, 28A′, 28A″) and a second strap (28B, 28B′, 28B″), and a release mechanism (30) coupled to the shell. The release mechanism includes a first cord (54A) coupled to the first strap and a second cord (54B) coupled to the second strap. The release mechanism is selectively moveable between a closed position and an open position.

### CROSS-REFERENCE TO RELATED APPLICATIONS

This patent application claims the priority benefit under 35 U.S.C. §119(e) of U.S. Provisional Application No. 61/416,317 filed on Nov. 23, 2010, the contents of which are herein incorporated by reference.

### BACKGROUND OF THE INVENTION

1. Field of the Invention

The present invention relates to respiratory therapy systems, such as non-invasive ventilation and pressure support systems, and, in particular, to a patient interface device for a respiratory therapy system that includes a quick release mechanism that allows the headgear component of the patient interface device to be quickly and easily loosened and tightened.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device, including a mask component having a soft, flexible cushion, on the face of a patient. The mask component may be, without limitation, a nasal mask that covers the patient's nose, a nasal cannula having nasal prongs that are received within the patient's nares, a nasal/oral mask that covers the nose and mouth, or a full face mask that covers the patient's face. Such patient interface devices may also employ other patient contacting components, such as forehead supports, cheek pads and chin pads. The patient interface device is connected to a gas delivery tube or conduit and interfaces the ventilator or pressure support device with the airway of the patient, so that a flow of breathing gas can be delivered from the pressure/flow generating device to the airway of the patient.

It is known to maintain such patient interface devices on the face of a wearer by means of a headgear component having one or more straps adapted to fit over/around the patient's head and attached to the mask using removable mask connectors. Such headgear components provide a means for adjustment of the patient interface device for various head shapes and allow a user to easily don or remove the patient interface device. Despite the presence of the removable mask connectors, many patients pull mask component of their patient interface device forward to remove it, stretching the headgear in the process component. Although there are undoubtedly many reasons that people do this, they do it despite having headgear connectors available for easy release or disengagement from the mask shell. The stretching of the headgear often adversely affect the fit and/or adjustment of the patient interface device, and thus is problematic and undesirable.

### SUMMARY OF THE INVENTION

Accordingly, it is an object of the present invention to provide a patient interface device that overcomes the shortcomings of conventional patient interface device. This object is achieved according to one embodiment of the present invention by providing a patient interface device that includes a cushion, a shell coupled to the cushion, a headgear having a first strap and a second strap, and a release mechanism coupled to the shell. The release mechanism includes a first cord coupled to the first strap and a second cord coupled to the second strap. The release mechanism is selectively moveable between a closed position and an open position.

In another embodiment, a method of using a patient interface device is provided that includes donning the patient interface device, wherein the patient interface device includes a cushion, a shell coupled to the cushion, a headgear having a first strap and a second strap, and a release mechanism coupled to the shell. The release mechanism includes a first cord coupled to the first strap and a second cord coupled to the second strap. The release mechanism is selectively moveable between a closed position and an open position. The method also includes adjusting a size of the headgear to a suitable size while the release mechanism is in the closed position and while the patient interface device is being worn, and placing the release mechanism into the open position and removing the patient interface device without further adjusting the size of the headgear.

In still another embodiment, a method of using a patient interface device is provided that includes choosing a particular non-adjustable headgear from among a plurality of differently sized non-adjustable headgears. The particular non-adjustable headgear includes a first strap and a second strap. The method further includes assembling the patient interface device by coupling the particular non-adjustable headgear to an assembly including a cushion, a shell coupled to the cushion, and a release mechanism coupled to the shell. The release mechanism includes a first cord coupled to the first strap and a second cord coupled to the second strap. The release mechanism is selectively moveable between a closed position and an open position. The method also includes donning the patient interface device while the release mechanism is in the open position, placing the release mechanism into the closed position, providing a breathing gas to the patient interface device while the patient interface device is being worn, placing the release mechanism into the open position and removing the patient interface device.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIGS. 1-6 are schematic diagrams (showing front and isometric views) of a system adapted to provide a regimen of respiratory therapy to a patient according to one exemplary embodiment of the present invention in various stages of operation;

FIGS. 7 and 8 are front and rear plan views, respectively, of a first interlocking member forming a part of a quick release mechanism of the system of FIGS. 1-6;

FIGS. 9 and 10 are front and rear plan views, respectively, of a second interlocking member forming a part of a quick release mechanism of the system of FIGS. 1-6;

FIG. 11 is a schematic diagram of a system adapted to provide a regimen of respiratory therapy to a patient according to an alternative exemplary embodiment of the present invention; and

FIG. 12 is a schematic diagram of a system adapted to provide a regimen of respiratory therapy to a patient according to an alternative exemplary embodiment of the present invention.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

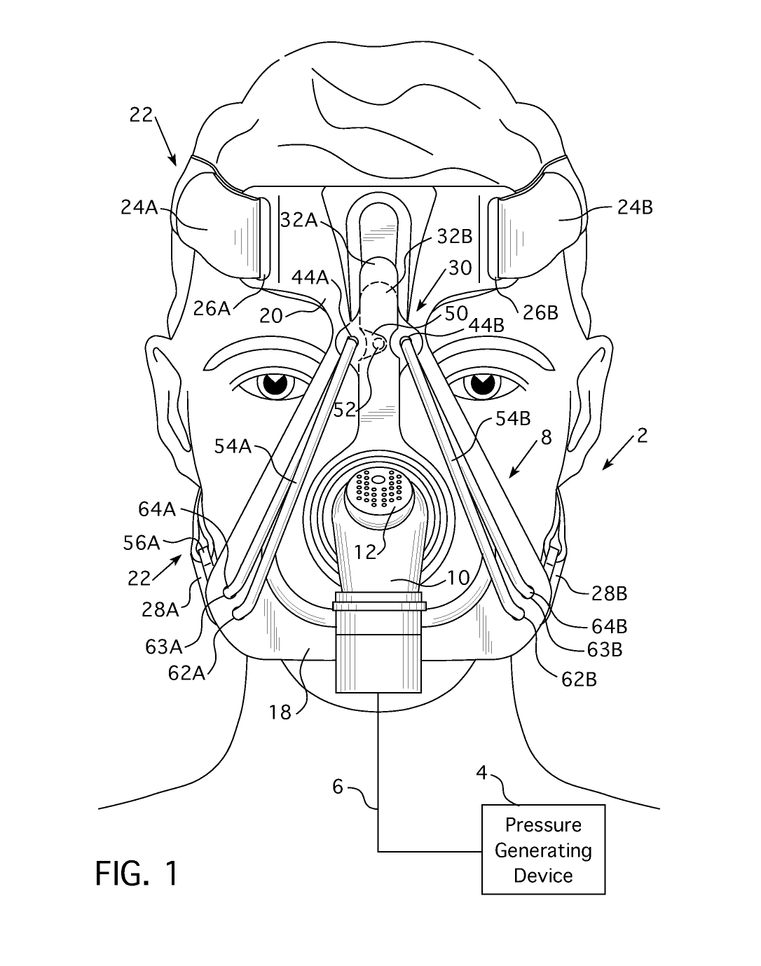

A system 2 adapted to provide a regimen of respiratory therapy to a patient according to one exemplary embodiment is generally shown in FIGS. 1-6 in various stages of operation. System 2 includes a pressure generating device 4, a patient circuit 6, a patient interface device 8, and an elbow conduit 10 having an exhaust port 12 provided therein. Although system 2 is discussed as including pressure generating device 4, patient circuit 6, patient interface device 8, and elbow conduit 10, it is contemplated that other systems may be employed while remaining within the scope of the present invention. For example, and without limitation, a system in which pressure generating device 4 is coupled to a patient interface device having an integrated exhaust port assembly is contemplated.

Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Patient circuit 6 is structured to communicate the flow of breathing gas from pressure generating device 4 to patient interface device 8. Patient interface 8 is typically a nasal or nasal/oral mask structured to be placed on and/or over the face of a patient. Any type of patient interface device 8, however, which facilitates the delivery of the flow of breathing gas to, and the removal of a flow of exhalation gas from, the airway of such a patient may be used while remaining within the scope of the present invention.

In the illustrated embodiment, patient interface 8 includes a cushion 16, a rigid shell 18, and a forehead support 20. Patient interface 8 also includes a headgear component 22 for securing patient interface device 8 to the patient's head. Headgear component 22 includes upper headgear straps 24A, 24B attached to shell 18 through slots 26A, 26B provided on opposite sides of forehead support 20. Headgear component 22 also includes lower headgear straps 28A, 28B that are moveably coupled to opposite sides of the lower portion of shell 18 in the manner described in greater detail elsewhere herein as part of the quick release mechanism of the present invention. An opening in shell 18 to which elbow conduit 10 is coupled allows the flow of breathing gas from pressure generating device 4 to be communicated to an interior space defined by shell 18 and cushion 16, and then, to the airway of a patient. The opening in shell 18 also allows the flow of exhalation gas (from the airway of such a patient) to be communicated to exhaust port 12 of elbow conduit 10 in the current embodiment.

Patient interface device 8 also includes a quick release mechanism 30 that allows headgear component 22 to be quickly and easily loosened and tightened to facilitate of donning and removal of patient interface device 8. In the exemplary embodiment, quick release mechanism 30 allows headgear component 22 to be loosened and tightened by a patient with as little as one hand and eliminates the need for the removable headgear connectors that are present in many current patient interface devices.

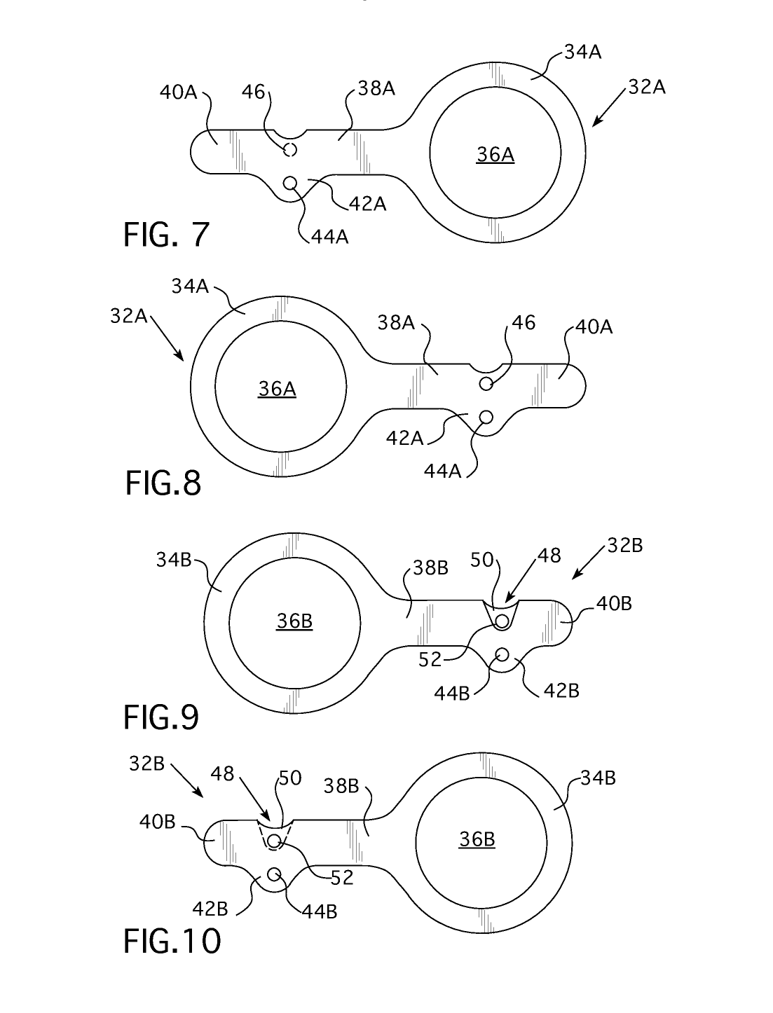

Quick release mechanism 30 includes a first interlocking member 32A and a second interlocking member 32B. As seen in FIGS. 7 and 8, in the exemplary embodiment, first interlocking member 32A is an elongated member that includes a circular base portion 34A defining an aperture 36A. An arm 38A extends from base portion 34A. A distal end 40A of first interlocking member 32A includes a protruding portion 42A having a hole 44A provided therein. The rear side of distal end 40A (FIG. 8) also includes a pin 46. As seen in FIGS. 9 and 10, in the exemplary embodiment, second interlocking member 32B is an elongated member that includes a circular base portion 34B defining an aperture 36B. An arm 38B extends from base portion 34B. A distal end 40B of second interlocking member 32B includes a protruding portion 42B having a hole 44B provided therein. The front side of distal end 40B also includes a lead in feature 48 including a ramp 50 leading to a hole 52. Together, pin 46 and lead in feature 48 including ramp 50, and hole 52 provide a pin and pivot arrangement for selectively interlocking first interlocking member 32A and second interlocking member 32B as described in greater detail elsewhere herein.

As seen in FIGS. 1-6, first interlocking member 32A and second interlocking member 32B are rotatably mounted on shell 18 by first inserting a cylindrical coupling member or hub that protrudes from the surface of shell 18 and which is structured to be fluidly coupled to elbow conduit 10 through aperture 36B of second interlocking member 32B. The hub is then inserted through aperture 36A of first interlocking member 32A. When this is done, first interlocking member 32A will rest on top of second interlocking member 32B, and both first interlocking member 32A and second interlocking member 32B will be able to rotate freely about the longitudinal axis of the hub. The significance of this free rotation is explained below.

Elbow conduit 10 is then coupled to the hub by a friction fit and, in the illustrated embodiment, both first interlocking member 32A and second interlocking member 32B are held in place and kept form falling off by a flange 19 attached to elbow conduit 10. In an alternative embodiment, the cylindrical hub includes four longitudinal slits around it outer circumference which enables the diameter thereof to be temporarily decreased by a compressive force so that the first interlocking member 32A and second interlocking member 32B can be slipped onto the hub. Thereafter, when the compressive force is removed, the hub will expand and the first interlocking member 32A and second interlocking member 32B will be prevented from falling off while still being able to rotate. Elbow conduit 10 in this embodiment may be provided with a similar split hub that is inserted into the shell hub and expands to hold it in place. Elbow conduit 10 may also be held in place by a split washer on the internal side of shell 18.

Furthermore, in the exemplary embodiment, quick release mechanism 30 also includes a cord 54A and a ring 56A and cord 54B and ring 56B. As seen in FIGS. 1-6, the end of lower headgear strap 28A is inserted through and coupled to ring 56A. In addition, in the exemplary embodiment, a first end 60A of cord 54A is coupled to ring 56A. Cord 54A is then inserted through a hole 62A provided on a first side of shell 18 (from the back of shell 18 toward the front of shell 18), passed upwardly along the front of shell 18, inserted through hole 44A of first interlocking member 32A, passed downwardly along the front of shell 18, and inserted back through a hole 63A (from the front of shell 18 toward the back of shell 18). A second end 64A of cord 54A opposite first end 60A is then attached to shell 18 at a location adjacent hole 63A. Similarly, as seen in FIGS. 1-3, the end of lower headgear strap 28B is inserted through and coupled to ring 56B. In addition, in the exemplary embodiment, a first end 60B of cord 54B is coupled to ring 56B.

Cord 54B is then inserted through a hole 62B provided on a second side of shell 18 (from the back of shell 18 toward the front of shell 18), passed upwardly along the front of shell 18, inserted through hole 44B of second interlocking member 32B, passed downwardly along the front of shell 18, and inserted back through a hole 63B (from the front of shell 18 toward the back of shell 18). A second end 64B of cord 54B opposite first end 60B is then attached to shell 18 at a location adjacent hole 63B. Once these steps are performed, construction of quick release mechanism 30 is complete.

In an alternative embodiment, rather than cords 54A, 54B extending downwardly along the front of shell 18 and being inserted back through a holes 63A, 63B as in the illustrated embodiment, they are fixedly attached to distal ends 40A, 40B after being passed upwardly along the front of shell 18.

In the exemplary, illustrated embodiment, cords 54A, 54B are inelastic, having essentially a fixed length. In an alternative embodiment, cords 54A, 54B may be elastic.

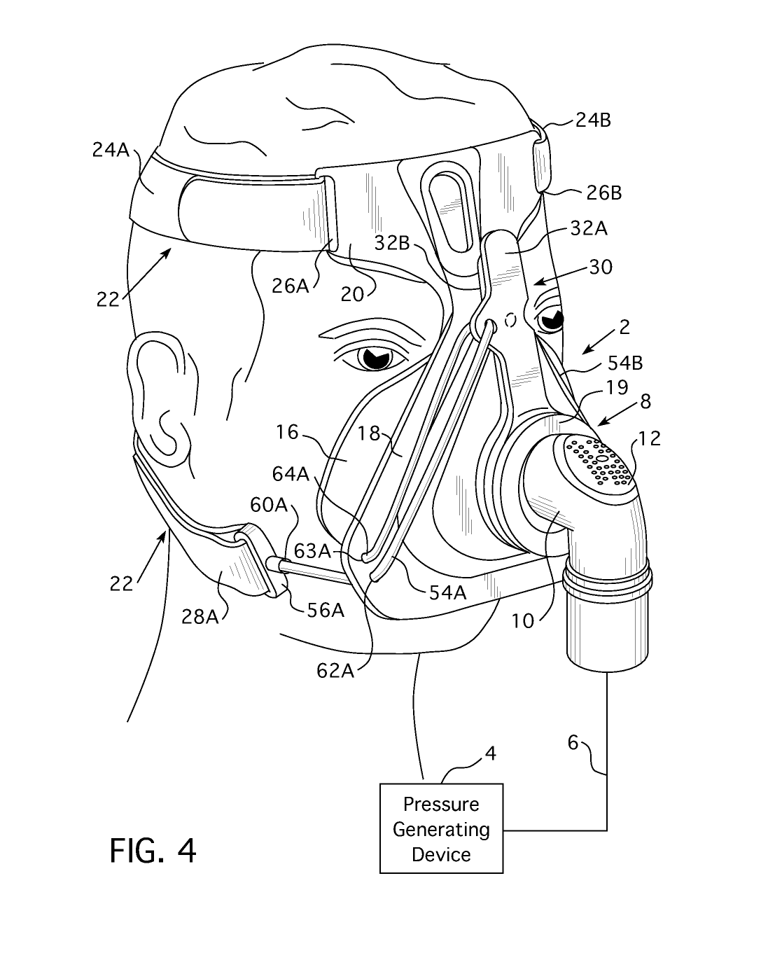

In operation, quick release mechanism 30 has two primary operating conditions/positions: (i) closed, and (ii) open. In the closed position, shown in FIGS. 1 and 4, first interlocking member 32A and second interlocking member 32B are vertically oriented and are interlocked to one another through operation of the pin and pivot arrangement described elsewhere herein. More specifically, in the closed position, pin 46 of first interlocking member 32A is received within hole 52 after having been lead to hole 52 by ramp 50. As shown in FIG. 4, in the closed position, the terminal end of each lower strap 28A, 28B is positioned no more than some predetermined minimum distance from shell 18, and as a result a maximum tension is applied to cords 54A, 54B, which pulls lower headgear straps 28A, 28B to provide a maximum tension thereto and thus provide a snug fit for the patient. The closed position is the position that quick release mechanism 30 is to be in when patient interface device 8 is to be used for respiratory therapy.

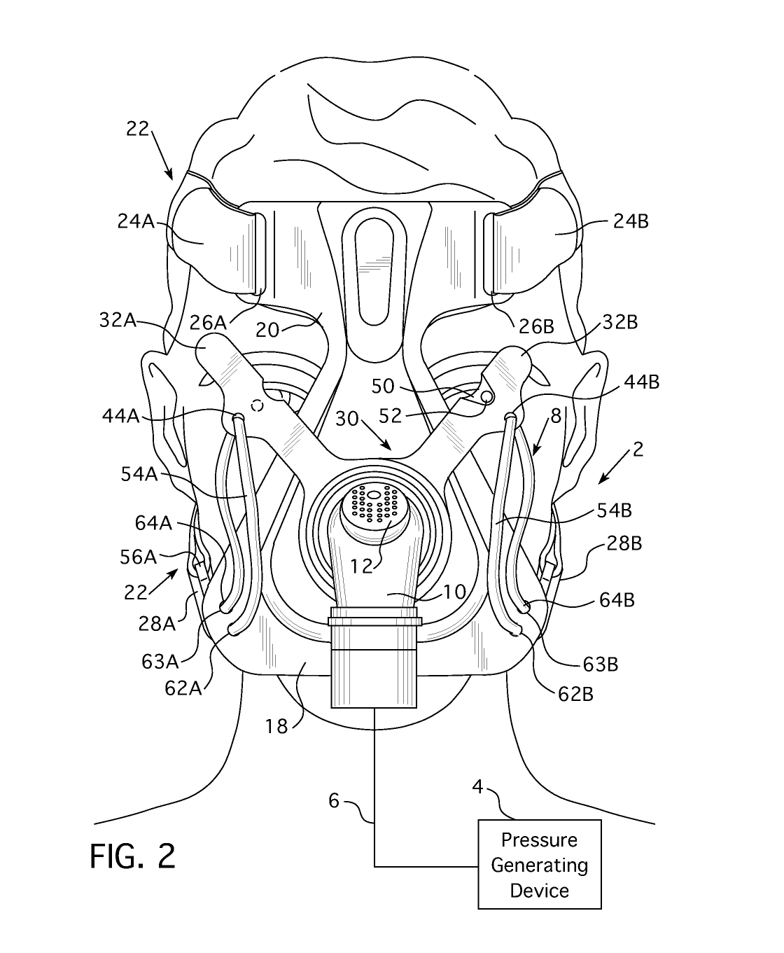

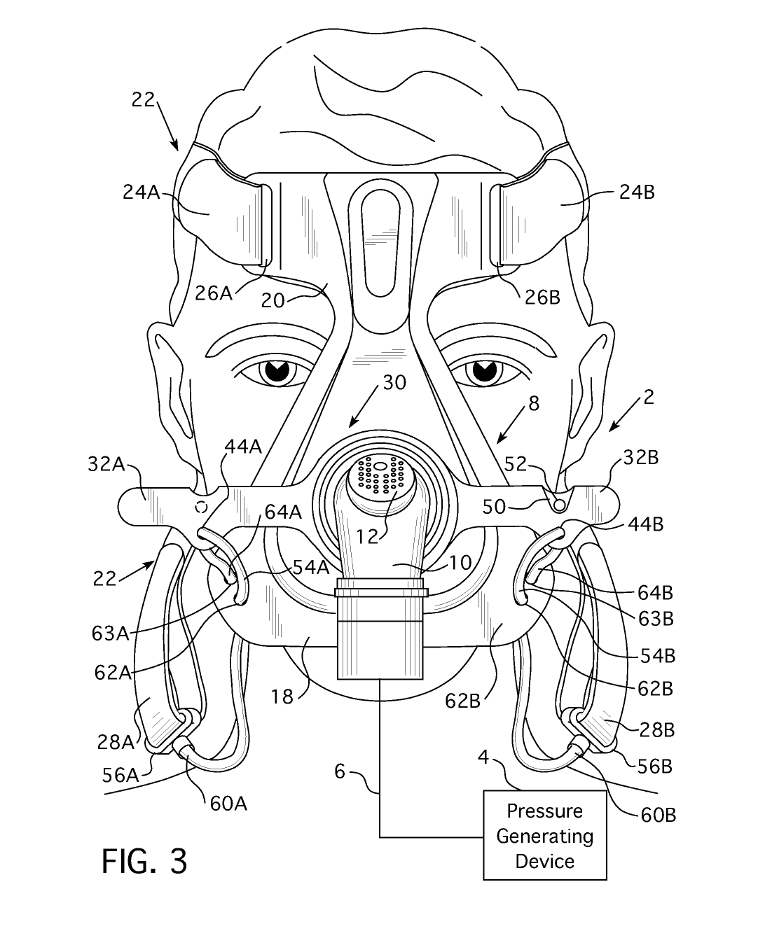

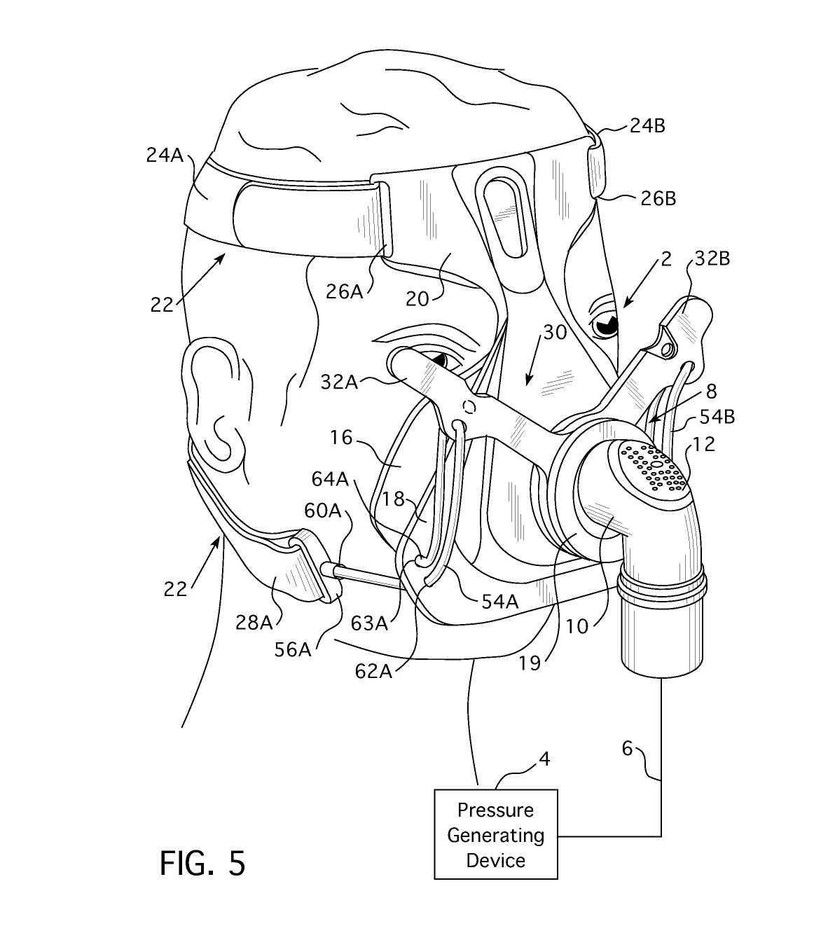

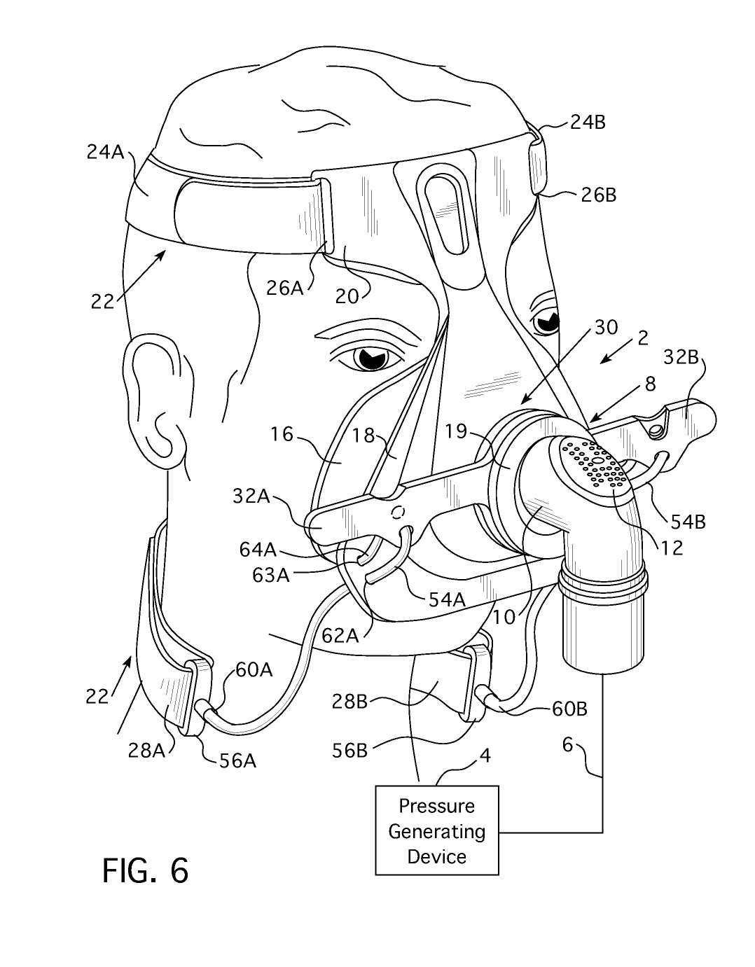

To get to the open position, first interlocking member 32A and second interlocking member 32B are released from one another by disengaging the pin and pivot arrangement. In the exemplary embodiment, the patient is able to do so by pulling on distal end 40A of first interlocking member 32A (which is longer than second interlocking member 32B), which releases pin 46 from hole 52. Once released from one another, first interlocking member 32A and second interlocking member 32B are rotated downwardly as shown in FIGS. 2 and 5 to the fully open position shown in FIGS. 3 and 6. In the exemplary, non-limiting embodiment, first interlocking member 32A and second interlocking member 32B are horizontally oriented in the fully open position.

As shown in FIGS. 3 and 6, in the fully open position, the tension on both cords 54A, 54B and on lower headgear straps 28A, 28B is released as a result of a portion of the length of cords 54A, 54B effectively being added to the length of lower headgear straps 28A, 28B. In the open position, the terminal end of each lower strap 28A, 28B is free to move beyond the predetermined minimum distance from shell 18. The patient may then freely and easily remove patient interface device 8 without pulling on shell 18 or stretching headgear 22. When the patient needs to don patient interface device 8 for therapy, the patient may place patient interface device 8 on his or her head with quick release mechanism 30 in the open position, position cushion 16 over his or her mouth and/or nose as appropriate, and then move quick release mechanism 30 to the closed position by rotating the first interlocking member 32A and second interlocking member 32B upwardly and interlocking them. When this is done, the lower headgear straps 28A, 28B will be pulled tight and headgear 22 will apply an appropriate, predetermined tension for an appropriate fit.

In the embodiment shown in FIGS. 1-6, the size of headgear component 22 is adjustable. In particular, the length of upper headgear straps 24A, 24B and lower headgear straps 28A, 28B is adjustable by any of a number of known methods. In the illustrated embodiment, headgear straps 24A, 24B and lower headgear straps 28A, 28B employ a hook and loop fastener system for this purpose wherein the exterior of each of the includes a loop fastener portion, and a corresponding hook fastener portion is provided on the exterior of each of end portions the straps. The strap may then be threaded through the associated ring or loop (26, 56) and then bent back on itself to adhere the hook fastener portion to the loop fastener portion when the desired length is achieved. Other suitable adjustment mechanisms such as, without limitation, a buckle arrangement commonly found on the straps of brassiere or a shoulder bag that allows the length of a strap to be adjusted may also be used. In this embodiment, the patient, a technician, or a patient and a technician together will preselect a suitable tension and fit for the patient interface device 8 while the quick release mechanism 30 is in the closed position (FIGS. 1 and 4) by adjusting upper headgear straps 24A, 24B and/or lower headgear straps 28A, 28B. Once this suitable tension and fit is determined, thereafter the patient need not alter headgear component 22, and instead may remove and don patient interface device 8 solely through operation of quick release mechanism 30.

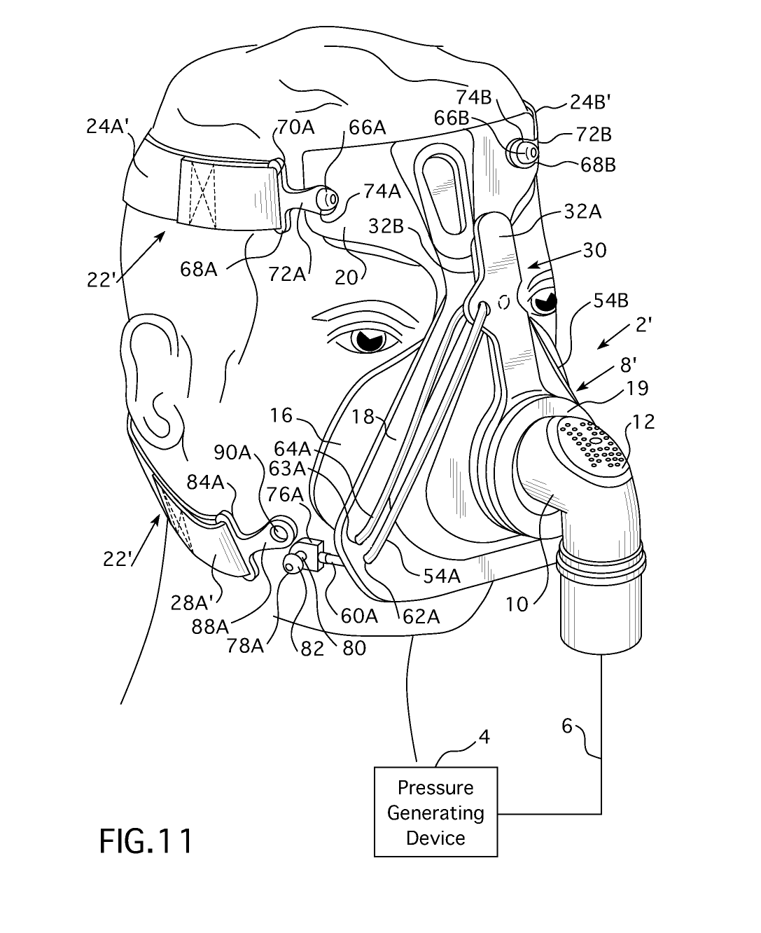

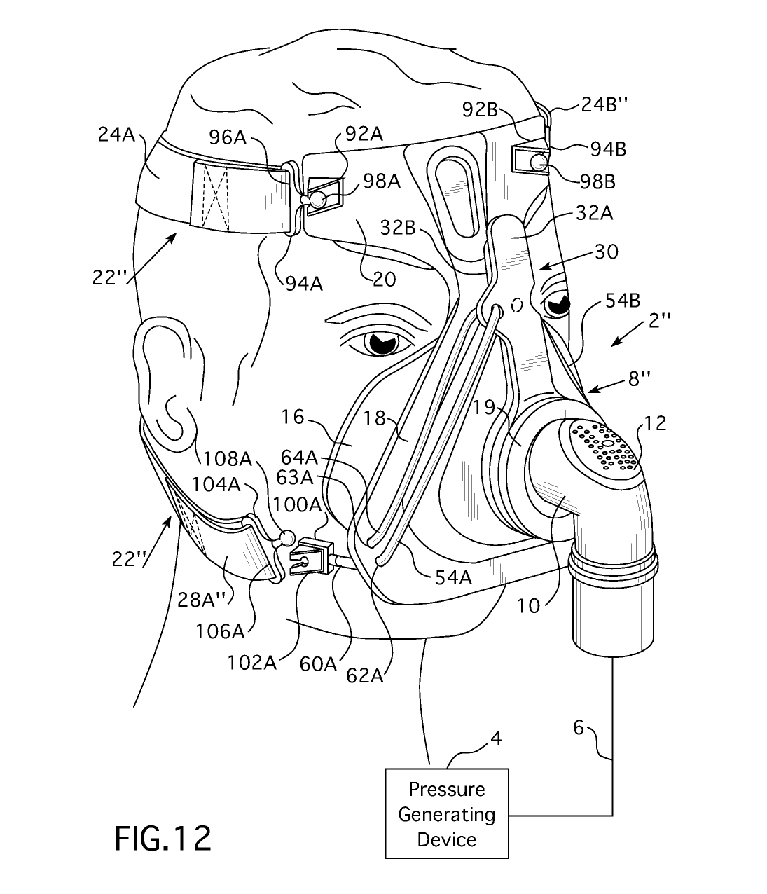

FIGS. 11 and 12 show systems 2′ and 2″, respectively, adapted to provide a regimen of respiratory therapy to a patient according to alternative embodiments of the present invention that include headgear components that are not freely adjustable (i.e., while lower headgear straps thereof may or may not stretch to an extent due to the material of which they are made, they are not provided with a mechanism that allows their length to be selectively adjusted). Systems 2′ and 2″ include many of the same components as system 2 described above, and thus like components are labeled with like reference numerals. Most notably, systems 2′ and 2″ each include a quick release mechanism 30 essentially as described elsewhere herein.

Referring to FIG. 11, system 2′ includes a patient interface 8′ having a headgear component 22′ for securing patient interface device 8′ to the patient's head. Headgear component 22′ includes upper headgear straps 24A′, 24B′, lower headgear strap 28A′ and a similar opposite lower headgear strap that is not shown, all of which are not freely adjustable in length as described above. Button elements 66A and 66B, each including a including a shaft and a cap, are provided are opposite sides of forehead support 20. A clip element 68A having a slot 70A and an arm 72A having an orifice 74A is attached to the end of upper headgear strap 24A′. Clip element 68A is made of a flexible material such that the portion thereof defining orifice 74A is structured to deform to pass over the cap of button element 66A and revert substantially to its original shape around and adjacent the shaft of button element 66A in order to connect clip element 68A to button element 66A and thus upper headgear strap 24A′ to forehead support 20. Operation of clip element 68B to couple to button element 66A and thus connect upper headgear strap 24B′ to forehead support 20 is similar.

Also referring to FIG. 11, first end 60A of cord 54A is connected to a headgear connector member 76A having a button element 78A including a shaft 80 and a cap 82. A clip element 84A having a slot 86A and an arm 88A having an orifice 90A is attached to the end of lower headgear strap 28A′. Clip element 84A is made of a flexible material such that the portion thereof defining orifice 90A is structured to deform to pass over cap 82 of button element 78A and revert substantially to its original shape around and adjacent 80 shaft of button element 78A in order to connect clip element 84A to button element 78A and thus lower headgear strap 28A′ to headgear connector member 76A and cord 54A. A similar headgear connector member and clip element are provided for connecting the other, opposite lower headgear strap to cord 54B.

Referring to FIG. 12, system 2″ includes a patient interface 8″ having a headgear component 22″ for securing patient interface device 8″ to the patient's head. Headgear component 22″ includes upper headgear straps 24A″, 24B″, lower headgear strap 28A″ and a similar opposite lower headgear strap that is not shown, all of which are not freely adjustable in length as described above. Socket elements 92A and 92B are provided are opposite sides of forehead support 20. A clip element 94A having a slot 96A and ball 98A is attached to the end of upper headgear strap 24A″. Ball 98A is structured to be received within socket element 92A in order to connect clip element 94A, and thus upper headgear strap 24A″, to forehead support 20. Operation of clip element 94B to couple to button element 66A and thus connect upper headgear strap 24B″ to forehead support 20 is similar.

Also referring to FIG. 12, first end 60A of cord 54A is connected to a headgear connector member 100A having a socket element 102A. A clip element 104A having a slot 106A and ball 108A is attached to the end of lower headgear strap 28A″. Ball 108A is structured to be received within socket element 102A in order to connect clip element 104A to socket element 102A and thus lower headgear strap 28A″ to headgear connector member 100A and cord 54A. A similar headgear connector member and clip element are provided for connecting the other, opposite lower headgear strap to cord 54B.

In the embodiments of FIGS. 11 and 12, a technician, or a patient and a technician together will preselect a suitable tension and fit for the patient interface device 8′, 8″ while the quick release mechanism 30 is in the closed position by selecting an appropriately sized headgear component 22′, 22″ from a number of differently sized non-adjustable headgear components 22′, 22″. Patients altering the size of a headgear determined to be appropriate for a patient by a technician can be problematic, as the altered size may adversely affect therapy. Thus, this embodiment is advantageous in that, once the size is selected, it cannot be altered by the patient.

It can be further appreciated that the present invention provides a mechanism that facilitates the removal and reattachment of a patient interface device while reducing the likelihood of stretching of the headgear component in the process.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface device, comprising:
a cushion;
a shell coupled to the cushion;
a headgear having a first strap and a second strap; and
a release mechanism coupled to the shell, the release mechanism having a first cord coupled to the first strap and a second cord coupled to the second strap, wherein the release mechanism is selectively moveable between a closed position and an open position, wherein the release mechanism includes a first elongate member rotatable relative to the shell about an axis through the shell and a second elongate member rotatable relative to the shell about the axis, wherein a first end of the first cord is coupled to the terminal end of the first strap and wherein the first cord is also coupled to the first elongate member, wherein a first end of the second cord is coupled to the terminal end of the second strap and wherein the second cord is also coupled to the second elongate member, wherein rotation of the first elongate member and the second elongate member about the axis in opposite directions causes the release mechanism to move from the closed position to the open position and releases a first tension on the first strap and a second tension on the second strap.

2. The patient interface device according to claim 1, wherein in the closed position a terminal end of the first strap is positioned no more than a first distance from the shell and a terminal end of the second strap is positioned no more than a second distance from the shell, and in the open position the terminal end of the first strap is free to move further than the first distance from the shell and the terminal end of the second strap is free to move further than the second distance from the shell.

3. The patient interface device according to claim 2, wherein the patient interface device is configured to cause the headgear to apply a first level of tension to a head of a patient when the patient interface device is donned by the patient and the release mechanism is in the closed position and to cause the headgear to apply a second level of tension to the head of the patient less than the first level when the patient interface device is donned by the patient and the release mechanism is in the open position.

4. The patient interface device according to claim 2, wherein when the release mechanism moves from the closed position to the open position and releases the first tension on the first strap and the second tension on the second strap, the terminal end of the first strap is allowed to move further than the first distance from the shell and the terminal end of the second strap is allowed to move further than the second distance from the shell.

5. The patient interface device according to claim 4, wherein in the closed position, the first elongate member and the second elongate member are positioned one on top of the other.

6-8. (canceled)

9. The patient interface device according to claim 4, further comprising an interlocking arrangement for interlocking the first elongate member and the second elongate member when the release mechanism is in the closed position.

10-12. (canceled)

13. The patient interface device according to claim 1, wherein the headgear includes a mechanism for selectively adjusting a size of the headgear.

14. (canceled)

15. A method of using a patient interface device, comprising:
(a) donning the patient interface device, the patient interface device including:
a cushion,
a shell coupled to the cushion,
a headgear having a first strap and a second strap, and
a release mechanism coupled to the shell, the release mechanism having a first cord coupled to the first strap and a second cord coupled to the second strap, wherein the release mechanism is selectively moveable between a closed position and an open position, wherein the release mechanism includes a first elongate member rotatable relative to the shell about an axis through the shell and a second elongate member rotatable relative to the shell about the axis, wherein a first end of the first cord is coupled to the terminal end of the first strap and wherein the first cord is also coupled to the first elongate member, wherein a first end of the second cord is coupled to the terminal end of the second strap and wherein the second cord is also coupled to the second elongate member, wherein rotation of the first elongate member and the second elongate member about the axis in opposite directions causes the release mechanism to move from the closed position to the open position and releases a first tension on the first strap and a second tension on the second strap;
(b) adjusting a size of the headgear to a suitable size while the release mechanism is in the closed position and while the patient interface device is being worn; and
(c) placing the release mechanism into the open position and removing the patient interface device without further adjusting the size of the headgear.

16. The method according to claim 15, wherein in the closed position a terminal end of the first strap is positioned no more than a first distance from the shell and a terminal end of the second strap is positioned no more than a second distance from the shell, and in the open position the terminal end of the first strap is free to move further than the first distance from the shell and the terminal end of the second strap is free to move further than the second distance from the shell.

17. The method according to claim 16, wherein the adjusting step also comprises placing the release mechanism into the closed position following the donning step.

18. The method according to claim 16, further comprising:
donning the patient interface device while the release mechanism is in the open position without further adjusting the size of the headgear;
placing the release mechanism into the closed position;
providing a breathing gas to the patient interface device while the patient interface device is being worn;
placing the release mechanism into the open position and removing the patient interface device without further adjusting the size of the headgear.

19. The method according to claim 18, wherein when the release mechanism moves from the closed position to the open position and releases the first tension on the first strap and the second tension on the second strap the terminal end of the first strap is allowed to move further than the first distance from the shell the terminal end of the second strap is allowed to move further than the second distance from the shell.

20.-23. (canceled)

24. A method of using a patient interface device comprising:
(a) choosing a particular non-adjustable headgear from among a plurality of differently sized non-adjustable headgears, the particular non-adjustable headgear having a first strap and a second strap;
(b) assembling the patient interface device by coupling the particular non-adjustable headgear to an assembly including:
a cushion,
a shell coupled to the cushion, and
a release mechanism coupled to the shell, the release mechanism having a first cord coupled to the first strap and a second cord coupled to the second strap, wherein the release mechanism is selectively moveable between a closed position and an open position, wherein the release mechanism includes a first elongate member rotatable relative to the shell about an axis through the shell and a second elongate member rotatable relative to the shell about the axis, wherein a first end of the first cord is coupled to the terminal end of the first strap and wherein the first cord is also coupled to the first elongate member, wherein a first end of the second cord is coupled to the terminal end of the second strap and wherein the second cord is also coupled to the second elongate member, wherein rotation of the first elongate member and the second elongate member about the axis in opposite directions causes the release mechanism to move from the closed position to the open position and releases a first tension on the first strap and a second tension on the second strap;
(c) donning the patient interface device while the release mechanism is in the open position;
(d) placing the release mechanism into the closed position;
(e) providing a breathing gas to the patient interface device while the patient interface device is being worn; and
(f) placing the release mechanism into the open position and removing the patient interface device.

25. The method according to claim 24, wherein in the closed position a terminal end of the first strap is positioned no more than a first distance from the shell and a terminal end of the second strap is positioned no more than a second distance from the shell, and in the open position the terminal end of the first strap is free to move further than the first distance from the shell and the terminal end of the second strap is free to move further than the second distance from the shell.

26. The method according to claim 25, wherein when the release mechanism moves from the closed position to the open position and releases the first tension on the first strap and the second tension on the second strap the terminal end of the first strap is allowed to move further than the first distance from the shell the terminal end of the second strap is allowed to move further than the second distance from the shell.

27-30. (canceled)
